(19) United States
(12) Patent Application Publication
(10) Pub. No.: US 2024/0230429 A1
(43) Pub. Date: Jul. 11, 2024

(54) THERMAL COMFORT MEASURING SYSTEM

(71) Applicant: HUKSEFLUX HOLDING B.V., Delft (NL)

(72) Inventor: Cornelis Jan VAN DEN BOS, Delft (NL)

(21) Appl. No.: 18/557,534

(22) PCT Filed: Apr. 28, 2022
(86) PCT No.: PCT/NL2022/050226

§ 371 Date: Oct. 26, 2023

(30) Foreign Application Priority Data
May 4, 2021 (NL) 2028142

(51) Int. Cl. G01K 17/00

(52) U.S. Cl. CPC G01K 17/00

(57) ABSTRACT

Disclosed is a heat flux sensor including a body with four or more sensor pairs, each pair including one radiation absorbing, absorptive, sensor for measuring a combined radiative and convective heat flux and one radiation reflecting, reflective, sensor for substantially measuring a convective heat flux, a heating member that is in heat conducing contact with the body, and a temperature sensor thermally coupled with the body for measuring the body temperature Tₛₑₙ.

### TECHNICAL FIELD

The invention relates to a heat flux sensor comprising a body with four or more sensor pairs, each pair consisting of one radiation absorbing sensor for measuring a combined radiative and convective heat flux and one radiation reflecting sensors for measuring a convective heat flux

The invention also relates to a system comprising such a heat flux sensor and to a method of determining a thermal comfort value, in particular a predicted mean vote (PMV).

### BACKGROUND

Thermal comfort is defined by ISO 7730 clause 7 as that condition of mind which expresses satisfaction with the thermal environment and is assessed using subjective evaluation. The 7730 standard presents methods to predict thermal sensation and degree of discomfort of people exposed to moderate thermal environments, like in homes, offices and cars. The methods are centered around the determination of the Predicted Mean Vote (PMV) and the associated Predicted Percentage of Dissatisfied (PPD). Knowing the PMV, the PPD can directly be calculated. One of the ways to determine the PMV is by direct measurement, using an integrating sensor. The concept of an integrating sensor refers to a measuring instrument performing the measurement of several variables needed to determine thermal comfort.

An important parameter in thermal comfort is the mean radiant temperature. It is defined as the uniform temperature of an imaginary enclosure in which radiant heat transfer from the human body is equal to the radiant heat transfer in the actual non-uniform enclosure.

ISO 7726 mentions in Clause 4.1.2 that the black globe thermometer is a device frequently used to derive an approximate value of the mean radiant temperature from the globe temperature, T_{g}, and the temperature and the velocity of the air surrounding the globe. It also points out that the measurement with the black globe is a major source of uncertainty of the PMV measurement. Large corrections need to be applied. These corrections depend on the air velocity, i.e. the estimate of the velocity of natural or forced convection, which needs to measured separately. In many cases the air velocity is not measured and thus not known, which leads to a larger measurement uncertainty than attainable with a known air velocity or direct heat flux measurements. A benefit of the globe thermometer is that it measures in all directions; it measures an integrated “mean” radiant temperature.

ISO 7726 clause 4.1.2 indicates that the spherical shape of the globe thermometer, integrating approximately over a solid angle of 4π sr, is a reasonable approximation of a seated person, but that an ellipsoidal shape would in fact be preferrable for the upright and seated positions.

In general ISO 7726 indicates that the mean radiant temperature may also be calculated from measured values if the plane radiant temperature in different directions is weighed according to the projected area factors for a person. In such case, multiple measurements are made with a sensor facing relevant directions, and the measured data are post-processed to arrive at the desired result. Calculating PMV for a certain situation, may require a procedure specific to the case, for example repositioning the sensor to point in different directions, and calculation based on scientific judgement, typically calculating an average or weighed average of the measurements, for example according to annex B of ISO 7726.

ISO 7726 further mentions in clause 4.1.2 that determining mean radiant temperature ideally requires measurements in 6 opposite directions and weighing these according to the projected area factors (optical view factors) for a person. Annex B on Mean Radiant Temperature comments on projected area factors in 6 directions (up-down, left-right, front-back, pairwise perpendicular axes) for thermal comfort estimation of persons in standing and seated positions. These projected area factors indicate the relative contribution of these directions for radiative exchange. Table B.1 of ISO 7726 shows that for a seated person all 6 directions are approximately equally relevant, while for a standing person the up-down directions make a smaller contribution than the others.

Although this is not specifically highlighted in ISO 7726, a measurement method of thermal comfort should also include a measurement of convective heat transfer representative of convective heat transfer to the human body. A common and reasonable assumption is that the projected area factors which are applicable to radiative heat flux are also valid for convective heat flux

A black globe thermometer or a ellipsoid body, ISO 7726 assumes, does not only exchange radiative energy but also convective energy in a way representative of the human body when seated. It seems reasonable to conclude that a thermal comfort sensor, capable of determining heat fluxes in one measurement, without repositioning, should have a view angle of substantially 2.5 π to 4π sr approximately equally sensitive in all 6 directions.

In US 2020/0189352, a vehicle with a thermal comfort sensor system is shown which includes an air velocity sensor, an air temperature sensor, a radiant heat flux sensor and a humidity sensor on one side of a headrest of a vehicle seat to sense the passenger compartment. Using this data, a climate controller calculates a current PMV-value of thermal comfort using the data from the sensor system and the data from a biometric sensor wearable by the passenger. A climate control system is further included for heating or cooling the exterior surface of the sensor system.

Operative temperature is defined as the uniform temperature in which an occupant would exchange the same amount of heat as in the existing non-uniform environment. Operative temperature is a practical way of expressing overall thermal exposure.

The invention aims to provide a reliable heat flux sensor and measurement system that can produce a more accurate determination of the heat fluxes at the sensor, that can provide an accurate operative temperature and that in particular can provide an accurate determination of the thermal comfort. It is also an object of the invention to produce a measurement immediately representative of the directional response of a human body in the situation that is investigated, taking projected area factors into account for persons in different positions without re-positioning the sensor. It is also an object of the invention to provide a versatile measurement system with which the need for separate air velocity measurements is obviated. It is a further aim to provide a sensor and a system in which the contribution of the different individual parameters that result in thermal comfort or discomfort, can be accurately determined.

### SUMMARY OF THE INVENTION

Hereto a heat flux sensor according to the invention comprises:
a body with four or more sensor pairs, each pair consisting of one radiation absorbing, absorptive, sensor for measuring a combined radiative and convective heat flux and one radiation reflecting, reflective, sensor for substantially measuring a convective heat flux,
the body exhibiting two or three axes of heat flux measurement, the axes being substantially perpendicular,
with two sensor pairs per axis facing in substantially opposite directions,
a heating member that is in heat conducing contact with the body, and
a temperature sensor thermally coupled with the body for measuring the body temperature Tₛₑₙ.

The sensor according to the invention is able to separately measure radiant and convective heat transfer from four or more sides of the sensor body, which may be made of metal. By its geometry it is capable of measuring heat transfer representative of that to a human body.

Typical shapes of the body are rectangular or cylindrical. In case of a rectangular body typically four or six sides are equipped with heat flux sensor pairs. In case of a cylindrical body, sensor pairs are typically deposited on 90° of the circular cross section and possibly on the top and bottom as well. A general requirement is that their combined field of view is between 2.5π to 4π sr.

The absorptive (typically black coated) heat flux sensor measures radiative as well as convective heat flux, whereas the reflective (typically gold-coated) heat flux sensor measures convective heat flux only. By subtracting the 2 measured fluxes, the radiative flux can be determined, the convective heat transfer coefficient Cₜᵣ can be determined and the ambient air velocity can be calculated. No separate air velocity measurement or estimate thereof is required. By separately determining all these factors, conclusions can be drawn about their contribution to the thermal comfort and about whether a specific factor is a cause or an effect.

The heat flux sensor also contains a body temperature sensor such as a thermocouple of thermistor and a heating member, such that the sensor can measure heat fluxes at multiple different sensor body temperatures. The body temperatures can be controlled to an estimated clothing temperature, which makes it possible to measure the exact heat loss at this clothing temperature. When local circumstances change, the temperature of the body can be adapted rapidly to the new conditions, contrary to the use of a known black globe sensor.

The heating member must have the capability of heating the sensor body to a substantially uniform temperature. For example, at 32° C. steady state body temperature at an air temperature between 15 and 20° C. and lower than 1 m/s air speed (typical office conditions), the temperature difference between sensors should remain below 2° C., so that 2 sensor pairs per axis facing in substantially opposite directions are substantially equally sensitive to radiative and convective heat flux. At heat flux level in the order of 100 W/m², and heat transfer coefficients combined radiative and convective heat flux in the order of 10 (W/m²)/C, a 2° C. error creates a 20% error, which is rated acceptable.

By measuring at more than 2 body temperature levels, the quality control and assurance of the thermal comfort measurements may be improved. Quality assurance of the heat flux measurements is also obtainable by comparing the total power in W required to keep the sensor at a stable temperature to the measured heat fluxes in W/m² and the sensor body surface area in m².

By first determining radiative and convective transfer coefficients, and then controlling the temperature of the body to a stable temperature, for example the clothing temperature, the operative temperature can be determined.

An embodiment of a heat flux sensor according to the invention comprises at least four differently oriented surfaces, each carrying a absorptive sensor and a reflective sensor.

By equipping multiple sides of the body with heat flux sensor pairs, the heat fluxes from different directions can be determined. By equipping the body with an even number of sensor pairs mounted with their fields of view in opposite directions, each having a field of view between 2π to 4π sr, imbalances or radiant and convective asymmetry can simultaneously be detected.

The measured heat fluxes, combined other measured parameters and parameters entered by the user, make it possible to calculate the PMV and PPD score for the different sides, in particular 4 or 6 sides, of the body and an overall integrated PMV and PPD score, which is based on the combined heat fluxes. A sensor for which the combined solid angle is between 2.5π sr and 4π sr may also act as an integrating sensor, approximating the solid angle and directional response of a human body. Results for seated and standing persons may also be calculated using mathematical weighing per axis as proposed by ISO 7726 Annex B.

The sensor body and sensors mounted on it may also be curved, for example cylindrical, having the same directional properties—that is sensitivity to radiative and convective fluxes—as an approximate cylindrical human body at the position of the sensor.

Using two sensor pairs mounted in opposite directions, in case of radiant or convective asymmetry, the sensor allows detection of such asymmetry and will be able to issue a warning, in a case in which the use of a known black globe thermometer could give to a “false positive” indication of a comfortable situation.

A system according to the invention comprises the heat flux sensor, a control unit and an ambient temperature sensor connected to the control unit for measuring an ambient temperature Tₐᵢᵣ, the control unit being adapted for:
operating the heating member for substantially uniform heating of the body,
determining or controlling of the temperature of the body, Tₛₑₙ,
determining from the measurements of the reflective sensor a convective heat flux Φ_{convection},
determining a radiative heat flux Φ_{radiation} by subtracting the measurements of the absorptive sensor and of the reflective sensor,
implementing corrections to improve measurements, for example correcting Φ_{convection}, and Φ_{radiation} case emission and reflection of sensor coatings deviate significantly from 1, implementing mathematical corrections such as the projected area factors valid for the person under study
determining a convective heat transfer coefficient Cₜᵣ based on Φ_{convection}, Tₐᵢᵣ and Tₛₑₙ or based on the power supplied to the heating member at several sensor body temperatures Tₛₑₙ, determining an ambient air velocity vₐᵢᵣ, based on the heat transfer coefficient Cₜᵣ and determining the radiative temperature T_{rad} from Tₛₑₙ and Φ_{radiation}

The system may have an active temperature control of the sensor body wherein the control unit controls the power supplied to the heating member so that the sensor body is kept at a predetermined temperature Tₛₑₙ.

In the system according to the invention, the heat transfer coefficient Cₜᵣ can be determined by

$$\\ {C}_{tr} = {Φ}_{convection} / ( {T}_{air} - {T}_{sen} ) ,$$

and the air velocity vₐᵢᵣ can be determined from Cₜᵣ, using an empirical formula valid for the sensor geometry and the direction of the ambient air flow, for example by:

$$\\ {v}_{air} = {( ( {C}_{tr} - B ) / A )}^{2}$$

wherein A and B are constants.

The constants A and B are typically empirically determined.

It is also possible to use average values of Φ_{convection} in several directions to determine the air velocity.

A correction may be made for non-perfect radiation absorption (i.e. absorption deviating from 1) and non-perfect reflection (i.e. reflection deviating from 1).

An embodiment of a system according to the invention further comprises a humidity sensor connected to the control unit for determining a humidity pₐ of the ambient air, the control unit being adapted for determining a comfort value for a person at the position of the sensor, based on:
the measured humidity pₐ of the ambient air,
the body temperature, Tₛₑₙ,
measured air temperature, Tₐᵢᵣ, and the
the radiative heat flux Φ_{radiation}, and the radiative and the convective heat flux Φ_{convection}.
a single measurement with a sensor having a representative directional response and field of view for the person at the position of the sensor or
a single measurement with a sensor not having a representative directional response and field of view for the person under study, and using mathematical corrections such as the projected area factors valid for the person under study
a series of measurements with a sensor not having a representative directional response and field of view for the person under study, repositioning the sensor to point in different directions, and calculating a result from these measurements based on scientific judgement

ISO 7730 expresses thermal comfort using the predicted mean vote, PMV, which is linked to the PPD index, predicted percentage dissatisfied. The PMV has a 7-point scale; hot, warm, slightly warm, neutral, slightly cool, cool and cold. From the PMV the predicted percentage dissatisfied, PPD, can be calculated.

The PPD has a minimum of 5% when PMV=0 (neutral) and reaches 75% when PMV=±2 (warm or cool). A person is considered to be dissatisfied when he or she considers the environment hot, warm, cool or cold.

In more detail: PMV is a function of 6 input parameters; metabolic rate M in W/m², clothing insulation I_{cl}, air temperature Tₐᵢᵣ, mean radiant temperature T_{rad}, relative air velocity vₐᵢᵣ and the water vapor partial pressure pₐ in Pa.

One standard metabolic unit (met) corresponds to 58.2 W/m². The metabolic rate for a sedentary activity is considered to be 1.2 met=70 W/m². The clothing insulation I_{cl} is expressed in K·m²/W. One standard clothing unit (clo) is 0.155 K·m²/W, which corresponds to a typical set of garment worn in a working environment.

According ISO 7730 the PMV can be calculated, within a certain rated operating range defining “moderate conditions”, most importantly vₐᵢᵣ <1 m/s, M<4 met, I_{cl}:<2 clo, Tₐᵢᵣ 10 to 30° C. by:

$$\\ PMV = [ 0.303 {e}^{{}_{} ( - 0.036 M )} + 0.028 ] \times [ \begin{matrix}[ ( M - W ) - 3.05 \times {10}^{- 3} [ 5733 - 6.99 ( M - W ) - {p}_{a} ] - \\ 0.42 [ ( M - W ) - 58.15 ] - 1.7 \times {10}^{- 5} M ( 5867 - {p}_{a} ) - 0.0015 M ( 34 - {T}_{air} ) - \\ 3.96 {10}^{- 8} {f}_{cl} ( {( {T}_{cl} + 273 )}^{4} - {( {T}_{rad} + 273 )}^{4} ) - {f}_{cl} {C}_{tr} ( {T}_{cl} - {T}_{air} )\end{matrix} ]$$

With T_{cl} the clothing temperature:

$$\\ {T}_{cl} = 35.7 - 0.028 ( M - W ) - {I}_{cl} [ 3.96 {10}^{- 8} {f}_{cl} ( {( {T}_{cl} + 273 )}^{4} - {( {T}_{rad} + 273 )}^{4} ) + {f}_{cl} {C}_{tr} ( {T}_{cl} - {T}_{air} ) ]$$

The second term between brackets in the equation for PMV represents the heat balance between the human being and its surrounding. If the balance is negative, this will result in a cool or cold sensation. The first five factors of the heat balance describe the heat loss by evaporation, by sweating and by respiration. Since the largest part of the human body is covered and insulated by clothing, the heat losses through radiation and convection are calculated at the clothing surface, using its estimated surface temperature, T_{cl}. The sixth and seventh component of the heat balance gives the linearized heat loss through radiation, the eight component the heat loss through convection. The radiation model assumes that 71% of the body area acts as a net emitter of radiative energy with an emissivity of 0.97. The remaining 29% of the area, for example between the legs, absorbs as much as it emits, resulting in a zero balance. These factors are included in the multiplier of 3.96 10⁻⁸ of the sixth component (0.97×0.71×5.77×10⁻⁸=3.96×10⁻⁸).

In this equation, T_{cl} is the clothing temperature, W the effective mechanical power, Cₜᵣ the convective heat transfer coefficient, f_{cl} clothing surface area factor: ratio of the surface area of the clothed body to the nude body. The effective mechanical power produced is usually set at 0 W, assuming office work or driving a car. This needs to be adapted if heavy work is done.

In a typical analysis, the user determines a typical metabolic rate, clothing surface area factor and the clothing insulation. As a next step T_{cl} must then be estimated.

In known systems, the black globe thermometer is a device frequently used to derive an approximate value of the mean radiant temperature from the globe temperature, T_{g}, and the temperature and the velocity of the air surrounding the globe. The measurement with the black globe is a major source of uncertainty of the PMV measurement that leads to large corrections being applied. These corrections depend on the air velocity, i.e. the estimate of the velocity of natural or forced convection, which is in the known systems usually not measured and thus not known. This inaccuracy is prevented with the system according to the invention.

Contrary to known black globe thermometer systems, and other systems described in ISO 7726 and 7730, the system according to the invention measures the heat loss through convection and radiation directly. Knowing the ratio between these two heat fluxes makes it possible to accurately estimate, using the above formula, the T_{cl}. This reduces the PMV measurement uncertainty relative to the crude estimate open to users of the black globe thermometer. The air velocity measurement or assumptions about air velocity are no longer needed; fluxes are directly measured.

In addition, the effect of projected area factors in 4 or 6 different directions for humans in different positions such as standing and seated, can be taken into account per surface in the terms (T_{rad}+273)⁴ and Cₜᵣ (T_{cl}−Tₐᵢᵣ).

A sensor for which the combined solid angle is between 2.5π sr and 4π sr may also act as an integrating sensor, approximating the solid angle and directional response of a human body. The known black globe thermometer, which has the shape and directional response of a sphere, integrates approximately over 4π Sr.

In an embodiment of the sensor, the sensor body and sensors mounted are example cylindrical, having the same directional properties—that is sensitivity to radiative and convective fluxes—as an approximately cylindrical human body under study.

In its preferred system embodiment, the sensor body temperature is controlled to the clothing temperature, and the PMV and PPD are determined with a sensor body temperature at or around T_{cl}.

It is possible to measure the PMV also if the body temperature of the measuring system is not at the calculated clothing (surface) temperature. For the measurement, we assume that the heat flux is a linear function of the body temperature and zero at ambient air temperature. At a small difference between actual and target temperature (a few degrees), the error in PMV is small.

Contrary to black globe thermometer systems, the present measuring system allows users to draw conclusions about cause and effect of discomfort. The black globe only issues a number. This discomfort is typically caused by unwanted cooling or heating of a particular part of the body; system users can separate the contributions of radiation, convection, and air velocity.

In an embodiment of a system according to the invention, the control unit is adapted to compare the total heating power supplied to the body for keeping the body at a predetermined temperature, with the heat flux measured by the reflective sensor and the heat flux measured by the absorptive sensor. This can provide a quality benchmark for the flux measurements.

In another embodiment of a system according to the invention, it measures Cₜᵣ and during the measurement the sensor body is controlled to a stable temperature, for example the appropriate clothing temperature Tal to accurately measure the convective and radiative flux at that temperature. This results in a high accuracy calculation of the operative temperature.

Alternatively, the operative temperature may be determined by the control unit heating the body at a number of different temperatures, and determining from the radiative heat fluxes radiation and the convective heat fluxes Φ_{convection} at these temperatures, and from those the radiative and convective heat transfer coefficients and an operative temperature.

In case the control unit determines a comfort value for different surfaces of the sensor body, mounted with their fields of view in opposite directions, it can provide an indication if the difference in comfort values measured at these surfaces is above a predetermined threshold value. In case of radiant or convective asymmetry, the sensor allows detection of such asymmetry and will be able to issue a warning.

The system according to the invention can also provide an indication, such as a visual sign or a sound or flagging of stored records, when the air velocity vₐᵢᵣ exceeds a predetermined threshold value. For accurate PMV measurements, it is preferred that the air velocity is below 1 m/s as air speeds above this value generally result in discomfort. The indication provided by the system prevents in this case that invalid PMV values are attributed.

### BRIEF DESCRIPTION OF THE DRAWINGS

FIG. 1 shows a perspective view of a sensor for measuring a heat flux in accordance with the present invention;

FIG. 2 shows a cross-sectional side view of a measurement side of the sensor for measuring a heat flux in accordance with the present invention;

FIG. 3 shows a perspective view of a measurement side of the sensor for measuring a heat flux in accordance with the present invention;

FIG. 4 shows a perspective view of a cylindrical sensor for measuring a heat flux in accordance with the present invention;

FIG. 5 shows a perspective view of a sensor for measuring a heat flux in accordance with the present invention;

FIG. 6 schematically shows a system according to the invention for measuring thermal comfort in accordance with the present invention;

FIG. 7 shows a flowchart of the steps carried out by the control unit for determining thermal comfort. in accordance with the present invention.

### DETAILED DESCRIPTION OF EMBODIMENTS

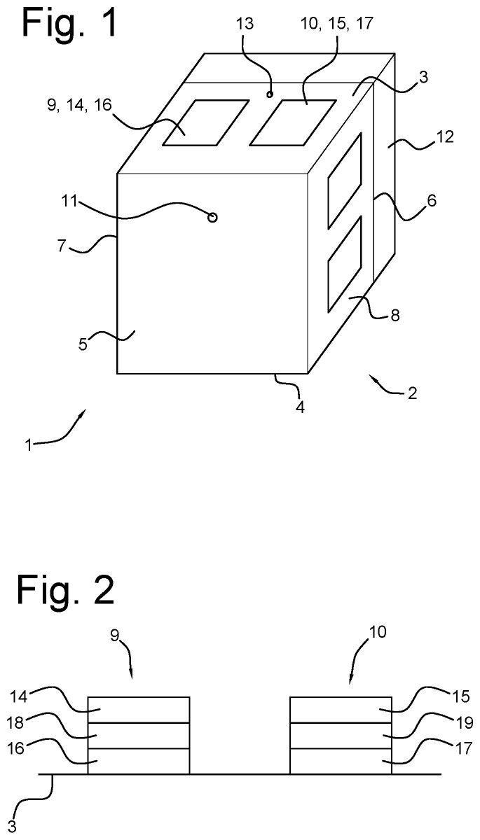

FIG. 1 shows a perspective view of a sensor 1 for measuring a heat flux in accordance with an embodiment of the present invention. The sensor comprises a body 2 with a plurality of sides 3, 4, 5, 6, 7, 8, an absorptive heat flux sensor 9 operable to absorb thermal radiation and a reflective heat flux sensor 10 operable to reflect thermal radiation. The sensor may optionally be equipped with an ambient air temperature sensor 13. The body 2 comprises at least one temperature sensor 11 for measuring the temperature of the body and at least one heating member 12. In this case the two sides 3 and 8 (not visible) each contain a sensor pair, the opposite sides 4 and 7 also contain a sensor pair.

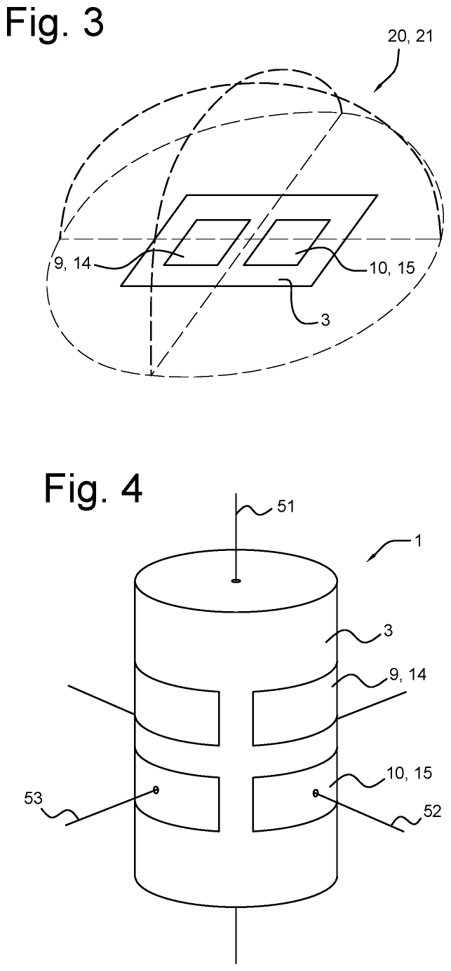

The absorptive heat flux sensor 9 operable to absorb thermal radiation is further referred to as absorptive sensor. This absorptive sensor 9 measures both convective and thermal radiation. The reflective heat flux sensor 10 operable to reflect thermal radiation is further referred to as reflective sensor. The reflective sensor 10 measures predominantly convective heat flux. The body 2 is well conducting heat, typically made of metal with a thermal conductivity of larger than 10 W/(m·K) and a heat capacity larger than 18 J/K typically made of metals such as aluminium, brass or steel. Each side of the body 3, 4, 5, 6, 7, 8 is a predominantly flat surface with a different spatial orientation. Four sides side 3, 4, 7, 8 of the body 2 are equipped with the absorptive sensor 9, the reflective sensor 10 and are further referred to as measurement sides 3, 4, 7, 8. The absorptive sensor 9 and the reflective sensor 10 on the measurement sides 3, 4, 7, 8 have a substantially similar mounting surface area and field of view as further described in FIG. 3, for the respective absorption and reflection of thermal radiation to be comparable and the sensitivity to convection to be comparable. The absorptive and reflective sensors may be placed anywhere on the measurement sides 3, 4, 7, 8, as long they have a substantially similar exposure of their surface area to thermal radiation and convection. The absorptive sensor 9 is typically black coated to absorb thermal radiation, the coating may be made of a black high temperature paint qualifying as high absorptivity layer 14 the heat flux sensor may be made using a thermopile sensing element encapsulated in a plastic 16, or another method measuring a temperature difference across a solid layer and presents a thermal absorptivity between 0.8 and 1. The absorptive sensor 9 measures both a convective and a radiative heat flux, Φ_{convection}+Φ_{radiation}, through its surface on the measurement side 3. The reflective sensor 10 is typically gold coated to reflect thermal radiation, may be made of a gold, silver, aluminium or nickel layer qualifying as high reflectivity layer 14 on a heat flux sensing element 17 and presents a thermal reflectivity between 0.5 and 1. Heat flux sensing elements 16, 17 convert the thermal energy of absorbed radiation for each sensor into an output signal, in case of a thermopile and electrical voltage. The reflective sensor 10 measures predominantly a convective flux Φ_{convection} through its surface on the measurement sides 3, 4, 7, 8.

By subtracting the heat flux measured by the reflective sensor 10, Φconvection, from the heat flux measured by the absorptive sensor 9, Φ_{convection}+Φ_{radiation}, through their surfaces of substantially equal sensitivity to convective heat flux on the measurement sides 3, 4, 7, 8, the radiative heat flux Φ_{radiation} may be estimated. The sensor 1 is therefore able to separately measure radiant and convective heat transfer on the measurement side 3. Mathematical corrections may be applied for reflectivity and absorptivity not equal to 1. The effect of projected area factors for humans in different positions such as standing and seated, can be taken into account per sensor side.

The heating member 12 is positioned in heat conducting contact with the body 2 and may be used to heat the body 2 to a predetermined temperature or at a predetermined power. Due to the body's high thermal conductivity, typically made of aluminium, brass or steel, combined with its mass, expressed as heat capacity, it internally has a low thermal resistance and the body is uniformly heated to the predetermined temperature, Tₛₑₙ, via heat conduction from the heating member.

The heating member must have the capability of heating the sensor body to a substantially uniform temperature, so that two sensor pairs per axis facing in substantially opposite directions are equally sensitive to radiative and convective heat flux.

The temperature sensor 11 thermally coupled with the body 2 is used to measure the temperature of the body. By controlling the body 2 to a predetermined temperature via the heating member 12 and the temperature sensor 11, radiative and convective heat transfer to an object of that particular temperature Tₛₑₙ, relevant to a user, can be simulated and measured. By measuring heat transfer using the absorptive sensor 9 and the reflective 10 sensor at multiple temperature levels of the body 2, from Φ_{convection, 1}, Φ_{convection, 2} at two or more predetermined temperatures T_{sen, 1}, T_{sen, 2}, the air temperature Tₐᵢᵣ and heat transfer coefficient Cₜᵣ can both be calculated, eliminating the need for a separate air temperature measurement, and improving the level of quality assurance of the heat transfer measurements:

$$\\ {C}_{tr} = ( {Φ}_{convection , 2} - {Φ}_{convection , 1} ) / ( {T}_{sen , 1} - {T}_{sen , 2} )$$

with positive heat flux from surrounding environment to the sensor, and/or

calculate an ambient air temperature Tₐᵢᵣ at or near the sensor by:

$$\\ {T}_{air} = {T}_{sen , 1} + {Φ}_{convection , 1} ( {T}_{sen , 1} - {T}_{sen , 2} ) / ( {Φ}_{convection , 2} - {Φ}_{convection , 1} )$$

The optional ambient air temperature sensor 13 can be also used to separately measure an air temperature. An ambient air velocity vₐᵢᵣ can further be calculated from the heat transfer coefficient using an empirical formula valid for the sensor geometry and the direction of the ambient air flow for example according to:

$$\\ {v}_{air} = {( ( {C}_{tr} - B ) / A )}^{2}$$

where A, B are constants empirically determined or taken from theory.

FIG. 2 shows a cross-sectional side view of the measurement plane 3, 4, 7 or 8 of the sensor 1 for measuring a heat flux of FIG. 1, according to an embodiment of the invention. In this embodiment, the absorptive sensor 9 comprises, besides the high absorptivity layer 14 and the thermopile sensing element 16, a calibration heating element 18. Furthermore the reflective sensor 10 comprises, besides the high reflectivity layer 15 and the heat flux sensing element 17, a calibration heating element 19.

Each calibration heating element 18, 19 is placed between the high absorptivity layer 14 and the thermopile sensing element 16 or between the high reflectivity layer 15 and the heat flux sensing element 17, respectively. Calibration heating elements 18, 19 may be identical and/or built integrally within the absorptive and reflective sensors 9, 10. The sensitivity, surface area and electrical resistance of the calibration heating elements 18, 19 are predetermined. They allow calculation of a measured calibration heat flux for a measured output voltage over or current through the calibration heating element.

By incorporating calibration heating elements 18, 19 on the heat flux sensors 9, 10, the sensors can be calibrated using a simple and unique procedure for both sensors, where the ratio of the measured calibration heat flux, Φ_{c}, to an output signal U of the thermopile or heat flux sensing element 16, 17 can be used to calculate a sensitivity E of the absorptive sensor and the reflective sensor 9, 10 according to:

$$\\ E = U / {Φ}_{c}$$

In other embodiments, alternative calibration functions may also be used. Heat fluxes may also be corrected for losses to the environment, edge effects etc. During calibration the heat flux sensor surfaces may be thermally insulated. The emissivity and absorption of the sensor surfaces may be separately estimated by inspection or by experiment.

FIG. 3 shows a perspective view of the measurement surface plane 3, 4, 7 or 8 of the sensor 1 for measuring a heat flux of FIG. 1 in accordance with an embodiment of the present invention. The predominantly flat measurement surface 3, 4, 7 or 8 is equipped with an absorptive sensor 9 and a reflective sensor 10 disposed at any location on the measurement surface and comprising the high absorptivity layer 14 and the high reflectivity layer 15, respectively, exposed to environmental heat fluxes. Each of the absorptive and reflective sensors 9, 10 have a substantially similar field of view 20, 21. Each field of view 20, 21 expands substantially 180 degrees in a longitudinal and in a transverse direction in an entrance plane at the measurement side 3, in other words a solid angle of 2π sr.

FIG. 4 shows a perspective view of a sensor body 1 according to an embodiment of the invention where the body is a cylinder and the sensors, 9, 10 comprising the high absorptivity layer 14 and the high reflectivity layer 15 are deposited on 90° of the circular cross section. In another embodiment they may as well be deposited the top and bottom of the cylinder. A general requirement is that their combined field of view is between 2.5π to 4π sr. The drawing also shows examples of pairwise perpendicular axes 51, 52, and 53 representing 6 directions of a person: up-down, left-right and front-back.

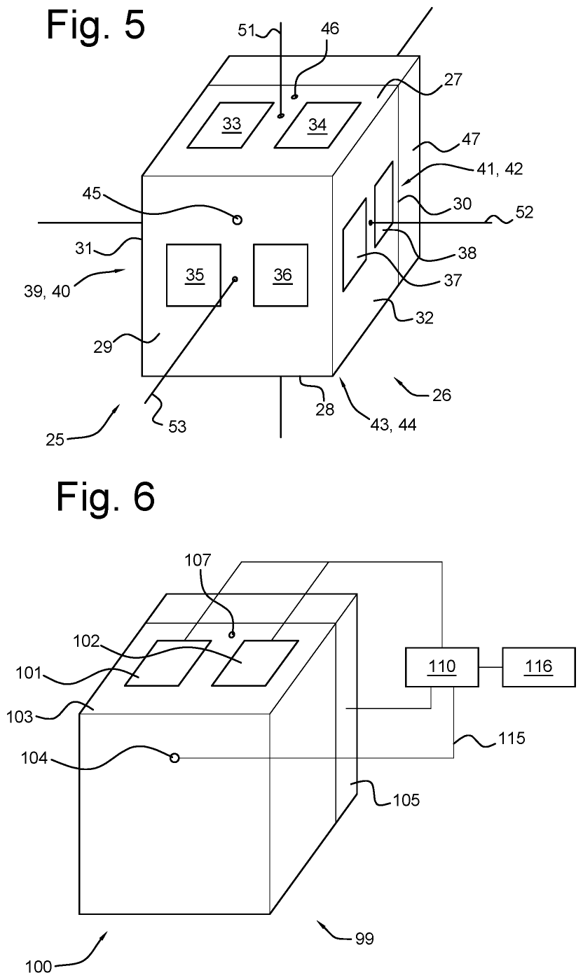

FIG. 5 shows a perspective view of a sensor 25 for measuring a heat flux in accordance with an embodiment of the present invention. The sensor comprises a body 26 with a plurality of sides 27, 28, 29, 30, 31, 32, at least four absorptive heat flux sensors 33, 35, 37, 39, 41, 43 operable to absorb thermal radiation and at least four reflective heat flux sensors 34, 36, 38, 40, 42, 44 operable to reflect thermal radiation. The sensor 25 may optionally be equipped with an ambient air temperature sensor 46. The body 26 comprises at least one body temperature sensor 45 and at least a heating member 47.

The at least four absorptive heat flux sensors 33, 35, 37, 39, 41, 43 operable to absorb thermal radiation are further referred to as absorptive sensors, and the at least four reflective heat flux sensors 34, 36, 38, 40, 42, 44 operable to reflect thermal radiation are further referred to as reflective sensors. They function identically and may be placed identically as the absorptive and reflective sensors described in the embodiment of FIG. 1, where each side 27, 28, 29, 30, 31, 32 is also a flat surface with a different spatial orientation. Further the absorptive sensor and the reflective sensor have also a substantially similar mounting plane and field of view as previously described in FIGS. 3 and 4. In the present embodiment at least two sides 27, 28, 29, 30, 31, 32 are each equipped with an absorptive sensor, a reflective sensor and are further referred to as measurement sides, for the respective sensitivities to convective heat flux and absorption and reflection of thermal radiation to be comparable. The sensors may also be calibrated with respective calibration heating element such as shown previously in FIG. 2.

The heating member 47 is positioned in heat conducting contact with the body 26 and may be used to heat the body 26 to a predetermined temperature, Tₛₑₙ. Due to the body's thermal conductivity, typically made of aluminium, brass or steel, and internally low thermal resistance the body is uniformly heated to the predetermined temperature via heat conduction from the heating member. The temperature sensor 45 thermally coupled with the body 26 may be used to measure the temperature of the body. By controlling the body 26 to a predetermined temperature via the heating member 47 and the temperature sensor 46, radiative and convective heat transfer to an object of that particular temperature with surfaces of different orientations 27, 28, 29, 30, 31, 32, relevant to a user, can be simulated and measured for each orientation.

By measuring heat transfer using the absorptive sensor 33, 35, 37, 39, 41, 43 and the reflective sensors 34, 36, 38, 40, 42, 44 at multiple temperature levels of the body 26, from the convective heat values Φ_{convection, 1}, Φ_{convection, 2} at two or more predetermined temperatures T_{sen, 1}, T_{sen, 2}, the air temperature Tₐᵢᵣ and heat transfer coefficient Cₜᵣ can both be calculated in the vicinity of each the at least two measurement sides, eliminating the need for a separate air temperature measurement, and improving the level of quality assurance of the heat transfer measurements, identically as in the embodiment of FIG. 1. Furthermore the ambient air velocity vₐᵢᵣ near each of the at least four measurement sides, can also be determined as in the embodiment of FIG. 1.

The drawing also shows examples of pairwise perpendicular axes 51, 52, and 53 representing 6 directions of a person: up-down, left-right and front-back.

FIG. 6 shows a system 99 for measuring thermal comfort, comprising a body 100, with the absorptive heat flux sensor 101 and the reflective heat flux sensor 102 situated on the same face 103 and having substantially similar fields of view between 1.5π and 3π sr. A temperature sensor 104, such as a thermocouple, for measuring the temperature of the body 100 is placed in heat conducting contact with the body 100. A control unit 110 is connected via control lines 115 to a heater 105 and temperature sensor 104 for heating the body 100 to a predetermined temperature, and to a temperature sensor and humidity sensor 107 for measuring the temperature and humidity of the ambient air. A terminal 116 is connected to the control unit 110. The terminal 116 comprises a user interface for the input of user commands and data to the control unit 110.

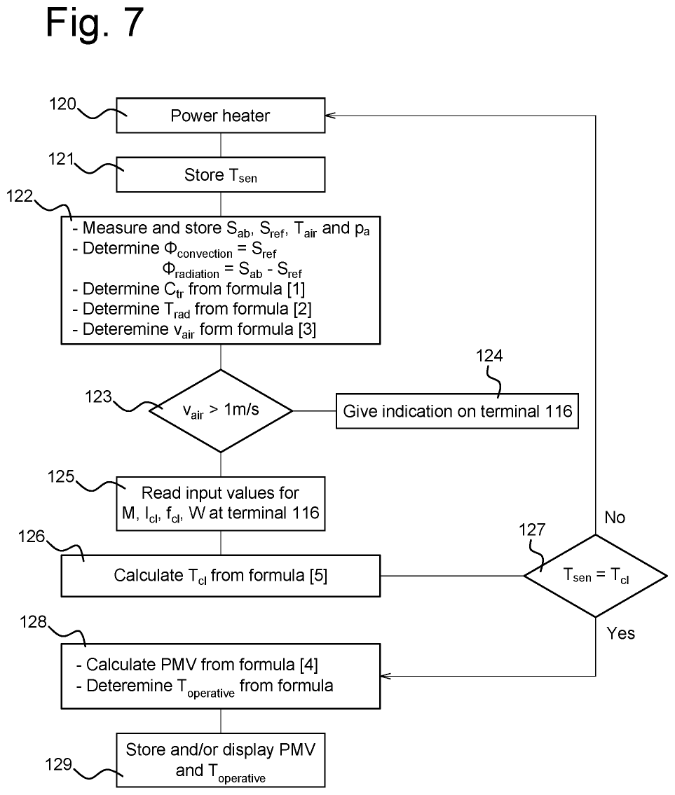

FIG. 7 shows a method of determining a thermal comfort value, in particular a predicted mean vote PMV. The control unit 110 comprises a processor and memory unit that calculate and store the PMV value and the operative temperature at the sensor body 100 by the following steps. The heater 105 is powered to set the temperature of the body 100 and sensors 101, 102 to a predetermined temperature, for example T_{cl} in step 120. The sensor temperature Tₛₑₙ is stored in step 121. In step 122:
the heat flux sensor signal S_{ab} of absorptive sensor 101 and the heat flux sensor S_{ref} of reflective sensor 102 are measured and stored in the memory of the control unit 110,
the air temperature signal Tₐᵢᵣ and the water vapour partial pressure signal pₐ of the humidity sensor are measured and stored in the memory of the control unit 110,
The convective heat flux Φ_{convection} is determined from the sensor value S_{ref} of reflective sensor 102: Φ_{convection}=S_{ref},
the radiative heat flux radiation is determined from the difference of the sensor value S_{ab} of the absorptive sensor 101 and the sensor value S_{ref} of the reflective sensor 102: Φ_{radiation}=S_{ab}−S_{ref},
the convective heat transfer coefficient of the air, Cₜᵣ is calculated by Formula 1, and
the air velocity vₐᵢᵣ is determined by Formula 5.

Corrections can be implemented in step 122 to improve measurements, for example correcting Φ_{convection}, and Φ_{radiation} in case the emission and reflection of sensor coatings deviate significantly from 1, or taking the effect of projected area factors for humans in different positions such as standing and seated, into account.

In step 123 it is determined if vₐᵢᵣ >1 m/s. If this is so, a digital, optical or acoustic warning signal is generated by the control unit 110 in step 124.

In step 125, the values for the metabolic rate, M, clothing isolation I_{cl}, clothing factor fa, the effective mechanical power W and a radiative heat transfer coefficient are entered by the user in the terminal 116 and read into the memory of the control unit 110.

In step 126:
the clothing temperature Ta is calculated by the control unit 110 by Formula 5,
the sensor body is controlled to Ta, as long as Tₛₑₙ is not within a pre-set limit from Tₛₑₙ, steps 120 to 125 may be repeated via step 127.

In step 128
the PMV value is calculated by Formula 4 and
the operative temperature, Tₒₚₑᵣₐₜᵢᵥₑ, is calculated by Formula 7.
radiant an convective asymmetries are calculated by subtracting heat flux values from sensors mounted with their fields of view in opposite directions.
a PMV may be calculated from a series of measurements possibly in different direction calculating a result from these measurements based on scientific judgement

The values of PMV and Tₒₚₑᵣₐₜᵢᵥₑ can be stored in the memory of the control unit 110 and/or displayed on a display of terminal 116 in step 129.

### Thermal Comfort

ISO 7730 clause 7 defines: Thermal comfort is that condition of mind which expresses satisfaction with the thermal environment and is assessed using subjective evaluation.

The 7730 standard presents methods to predict thermal sensation and degree of discomfort of people exposed to moderate thermal environments, like in homes, offices and cars. The methods are centered around the determination of the Predicted Mean Vote (PMV) and the associated Predicted Percentage of Dissatisfied (PPD). One of the ways to determine the PMV, described in clause 4.1, is by direct measurement, using an integrating sensor.

ISO 7726 mentions in clause 4.1.2 that determining heat fluxes ideally requires measurements in 6 opposite directions and weighing these according to the projected area factors (optical view factors) for a person. Annex B on Mean Radiant Temperature comments on projected area factors in 6 directions (up-down, left-right, front-back, pairwise perpendicular axes) for thermal comfort estimation of persons in standing and seated positions. These projected area factors indicate the relative contribution of these directions for radiative exchange. Table B.1 of ISO 7726 shows that for a seated person all 6 directions are approximately equally relevant, while for a standing person the up-down directions make a smaller contribution than the others.

ISO 7726 mentions in Clause 4.1.2 that the black globe thermometer is a device frequently used to derive an approximate value of the mean radiant temperature from the globe temperature, T_{g}, and the temperature and the velocity of the air surrounding the globe. It also points out that the measurement with the black globe is a major source of uncertainty of the PMV measurement. Large corrections are applied. These corrections depend on the air velocity, i.e. the estimate of the velocity of natural or forced convection, which is usually not measured and thus not known, which leads to a larger measurement uncertainty than attainable with a known air velocity or direct heat flux measurements.

Instead of black globe sensor, the present invention provides a new thermal comfort measuring system 99 based on measurement of radiative and convective heat fluxes with at least four sensor pairs such as 101, 102 to the temperature controlled sensor body 100.

The thermal comfort measuring system is able to separately measure radiant and convective heat transfer from one or more sides of a (metal) heat transfer sensor body 100. To do so, at least four sides of the sensor body 100 is equipped with the heat flux sensor pair such as 101, 102: an absorptive (typically black coated) heat flux sensor 101, which measures radiative as well as convective heat flux, and a reflective (typically gold-coated) heat flux sensor 102, which measures convective heat flux only. By subtracting the 2 fluxes measured by the sensors 101,102 in the control unit 110, the radiative flux may be estimated.

Preferably, the temperature of the sensor body 100 is controlled by means of the heater 106, temperature sensor 104 and the control unit 110, so that the system 99 can measure heat fluxes at the sensor pair 101, 102 at multiple different body temperatures.

The heating member must have the capability of heating the sensor body to a substantially uniform temperature. For example, at 32° C. steady state body temperature at an air temperature between 15 and 20° C. and lower than 1 m/s air speed (typical office conditions), the temperature difference between sensors should remain below 2° C., so that two sensor pairs per axis facing in substantially opposite directions are equally sensitive to radiative and convective heat flux.

The heat fluxes in W/m², the body temperature in ° C., possibly combined with the power in W supplied to the heater 105 and needed to maintain a fixed body temperature, and the ambient air temperature measured by the sensor 107 in ° C., are used in the control unit 110 to calculate all local parameters determining thermal transport, the fluxes caused by radiation and convection and the heat transfer coefficient.

In combination with a temperature and humidity measurement of the ambient air in % measured by the sensor 107, and user-entered parameters specific to the situation that is investigated the system 99 according to the invention can be used to predict thermal sensation and degree of discomfort (PMV and PPD) as described by ISO 7730, ASHRAE Standard 55 and CEN CR 1752. The complete system is then considered an “integrating sensor to directly measure the thermal comfort” as described in ISO 7730, paragraph 4.1.

When measuring at a known sensor body temperature, if the ambient air temperature is also known, or from measurements at multiple body temperatures, the local heat transfer coefficient can be estimated. From the heat transfer coefficient, the local air velocity can be calculated.

The system 99 can also be used to directly measure operative temperature. This can be done by controlling the sensor body 100 to a stable temperature, for example the clothing temperature and using the measured heat flux values at that temperature, or by calculation after performing multiple measurements at different sensor body temperatures, measured by the sensor 104 and set in the control unit through powering of the heater 105. If needed the radiative and convective transfer coefficients can be corrected to account for the properties of the “occupant”, resulting in an adapted estimate.

### System Physics

The black coated heat flux sensor 101 measures Φ_{radiation}+Φ_{convection}. The gold coated heat flux sensor 102 measures Φ_{convection} only. By subtraction, radiation can be calculated.

The sensor body can be operated at a certain constant power in W or be controlled to a certain body temperature in ° C.

The convective heat transfer coefficient of the air, with positive heat fluxes from surrounding environment (air) to the sensor, is calculated by:

$${C}_{tr} = {Φ}_{convection} \text{/(} {T}_{air} - {T}_{sen} ) \qquad [ 1 ]$$

The radiative temperature is:

$${{T}_{rad} = ( {T}_{sen}^{4} + {Φ}_{radiative} \text{/(} ε σ ) )}^{1 / 4} \qquad [ 2 ]$$

with & the absorption of the sensor coating and σ the Stefan Boltzmann constant.

The ambient air velocity can be estimated, from Cₜᵣ using [1] and empirical formula's such as:

$${V}_{air} = {( ( {C}_{tr} - B ) / A )}^{2} \qquad [ 3 ]$$

In such empirical formula's the constants A and B may be empirically determined. Typical orders of magnitude for a single perpendicularly exposed heat flux sensor surface: A=36.7 and B=−1.25. For surfaces exposed to air flow at 45°: A=30. 0 and B=−0.78.

It is also possible to use average values of Φ_{convection} under several directions relative to the airflow to determine the air velocity.

The electrical power consumption of the heater 106 at one or more body temperature levels of the body 100, may also be used to estimate the heat transfer coefficient.

### PMV, PPD Measurements

ISO 7730 expresses thermal comfort using the predicted mean vote, PMV, which is linked to the PPD index, predicted percentage dissatisfied. The PMV has a 7-point scale; hot, warm, slightly warm, neutral, slightly cool, cool and cold. From the PMV the predicted percentage dissatisfied, PPD, can be calculated. The PPD has a minimum of 5% when PMV=0 (neutral) and reaches 75% when PMV=±2 (warm or cool). A person is considered to be dissatisfied when he or she considers the environment hot, warm, cool or cold.

In more detail: PMV is a function of 6 input parameters; metabolic rate M in W/m², clothing insulation I_{cl}, air temperature Tₐᵢᵣ, mean radiant temperature T_{rad}, relative air velocity vₐᵢᵣ and the water vapour partial pressure pₐ in Pa.

One standard metabolic unit (met) corresponds to 58.2 W/m². The metabolic rate for a sedimentary activity is considered to be 1.2 met=70 W/m². The clothing insulation I_{cl} is expressed in K·m²/W. One standard clothing unit (clo) is 0.155 K·m²/W, which corresponds to a typical set of garment worn in a working environment.

According ISO 7730, the PMV can be calculated, within a certain rated operating range defining “moderate conditions”, most importantly vₐᵢᵣ <1 m/s, M<4 met, I_{cl}:<2 clo, Tₐᵢᵣ 10 to 30° C.

$$P M V = [ 0.303 {e}^{( - 0.036 M )} + 0.028 ] \times [ [ ( M - W ) - 3.05 \times {10}^{- 3} [ 5733 - 6.99 ( M - W ) - {p}_{a} ] - 0.42 [ ( M - W ) - 58.15 ] - 1.7 \times {10}^{- 5} M ( 5867 - {p}_{a} ) - 0.0015 M ( 34 - {T}_{a i r} ) - 3.96 {10}^{- 8} {f}_{cl} ( {( {T}_{cl} + 273 )}^{4} - {( {T}_{r a d} + 273 )}^{4} ) - {f}_{cl} {C}_{t r} ( {T}_{cl} - {T}_{a i r} ) ] \qquad [ 4 ]$$

The second term represents the heat balance between the human being and its surrounding. If the balance is negative, this will result in a cool or cold sensation. The first and second lines of the heat balance describe the heat loss by evaporation, by sweating and by respiration.

Since the largest part of the human body is covered and insulated by clothing, the heat losses through radiation and convection are calculated at the clothing surface, using its estimated surface temperature, T_{cl}.

The third component of the heat balance gives the linearized heat loss through radiation, the fourth component the heat loss through convection. The radiation model assumes that 71% of the body area acts as a net emitter of radiative energy with an emissivity of 0.97. The remaining 29% of the area, for example between the legs, absorbs as much as it emits, resulting in a zero balance. These factors are included in the multiplier of 3.96 10⁻⁸ of the third component (0.97×0.71×5.77×10⁻⁸=3.96×10⁻⁸). In this equation, T_{cl} is the clothing temperature, W the effective mechanical power, Cₜᵣ the convective heat transfer coefficient, f_{cl} clothing surface area factor: ratio of the surface area of the clothed body to the nude body. The effective mechanical power produced is usually set at 0 W, assuming office work or driving a car. This needs to be adapted (through user input via the terminal 116) if heavy work is done.

In a typical analysis, the user determines a typical metabolic rate, clothing surface area factor and the clothing insulation. These data are entered into the control unit 110 via the terminal 116. As a next step, in the control unit 110, T_{cl} must then be estimated by iteration, using the 3.96 factor mentioned above again to correct radiative view factors:

$${T}_{cl} = 3 5.7 - 0.028 ( M - W ) - {I}_{cl} [ 3.96 {10}^{- 8} {f}_{cl} ( {( {T}_{cl} + 273 )}^{4} - {( {T}_{r a d} + 273 )}^{4} ) + {f}_{cl} {C}_{t r} ( {T}_{cl} - {T}_{a i r} ) ] \qquad [ 5 ]$$

Operative temperature Tₒₚₑᵣₐₜᵢᵥₑ is defined in ISO 7730 as: the uniform temperature of an imaginary black enclosure, and the air within it, in which an occupant would exchange the same amount of heat by radiation plus convection as in the actual nonuniform environment.

In ISO 7726, appendix G2 gives the exact equation for a sensor for which the directional response is representative of the human body, or using a series of measurements to determine a representative heat flux Measuring Φ_{radiation, cl} and Φ_{convection, cl}, using a sensor with its body stabilised at one or more temperatures for example T_{cl}, allows accurate calculation of Tₒₚₑᵣₐₜᵢᵥₑ:

$${T}_{operative} = {T}_{cl} + ( {Φ}_{radiation , cl} + {Φ}_{convection , cl} \text{)/(} {C}_{tr} + {C}_{rad} ) \qquad [ 7 ]$$

1. Heat flux sensor comprising:
a body with six or more sensor pairs, each pair consisting of one radiation absorbing, absorptive, sensor for measuring a combined radiative and convective heat flux and one radiation reflecting, reflective, sensor for substantially measuring a convective heat flux,
the body exhibiting three axes of heat flux measurement, the axes being substantially perpendicular, with two sensor pairs per axis facing substantially in opposite directions, a heating member that is in heat conducing contact with the body, capable of heating the body uniformly within 2° C. for body temperatures between 25 and 40° C. at ambient air temperatures between 20 and 25° C. at air speeds <1 m/s and a temperature sensor thermally coupled with the body for measuring the body temperature Tₛₑₙ.

2. The heat flux sensor according to claim 1, carrying six or more sensor pairs with a combined field of view between 2.5π to 4π sr,

3. System comprising the heat flux sensor of claim 1, a control unit and an ambient temperature sensor connected to the control unit for measuring an ambient temperature Tₐᵢᵣ, the control unit being adapted for:
operating the heating member for heating of the body,
determining or controlling of the temperature of the body, Tₛₑₙ
determining from the measurements of the reflective sensor of each of the six or more sensor pairs a convective heat flux Φ_{convection},
determining a radiative heat flux Φ_{radiation} by subtracting the measurements of the absorptive sensor and of the reflective sensor of each of the six or more sensor pairs,
determining a convective heat transfer coefficient Cur based on Φ_{convection}, Tₐᵢᵣ and Tₛₑₙ or based on the power supplied to the heating member at several sensor body temperatures Tₛₑₙ,
determining an ambient air velocity vₐᵢᵣ, based on the heat transfer coefficient Cₜᵣ, and
determining the radiative temperature T_{rad} from Tₛₑₙ and Φ_{radiation}.

4. The system according to claim 3, wherein the control unit controls the power supplied to the heating member so that the sensor body is kept at a predetermined temperature Tₛₑₙ.

5. The system according to claim 3, further comprising a humidity sensor connected to the control unit for determining a humidity pₐ of the ambient air, the control unit being adapted for determining a comfort value for a person at the position of the sensor, based on:
the measured humidity pₐ of the ambient air,
the measured air temperature, Tₐᵢᵣ,
the body temperature, Tₛₑₙ,
for each of the six or more sensor pairs, the radiative heat flux Φ_{radiation}, and the convective heat flux Φ_{convection}.
and using mathematical corrections such as projected area factors valid for a person at the position of the sensor.

6. The system according to claim 5, wherein the comfort value comprises a predicted mean vote PMV which is a function of parameters including a metabolic rate M, that can be expressed in W/m², the effective mechanical power W, that can be expressed in W/m², clothing insulation I_{cl}, that can be expressed in K·m²/W, the air temperature Tₐᵢᵣ, a mean radiant temperature T_{rad}, that can be expressed in C, the ambient air velocity vₐᵢᵣ, that can be expressed in m/s and the measured humidity pₐ that can be expressed by water vapor partial pressure in Pa, which parameters are related:
$$P M V = [ 0.303 {e}^{( - 0.036 M )} + 0.028 ] \times [ [ ( M - W ) - 3.05 \times {10}^{- 3} [ 5733 - 6.99 ( M - W ) - {p}_{a} ] - 0.42 [ ( M - W ) - 58.15 ] - 1.7 \times {10}^{- 5} M ( 5867 - {p}_{a} ) - 0.0015 M ( 34 - {T}_{a i r} ) - 3.96 {10}^{- 8} {f}_{cl} ( {( {T}_{cl} + 273 )}^{4} - {( {T}_{r a d} + 273 )}^{4} ) - {f}_{cl} {C}_{t r} ( {T}_{cl} - {T}_{a i r} ) ] ,$$
Wherein values of M, W, I_{cl}, f_{cl} are entered by the user, and f_{c}l is a clothing surface area factor, and T_{cl} the clothing surface temperature:
$${T}_{cl} = 3 5.7 - 0.028 ( M - W ) - {I}_{cl} [ 3.96 {10}^{- 8} {f}_{cl} ( {( {T}_{cl} + 273 )}^{4} - {( {T}_{r a d} + 273 )}^{4} ) + {f}_{cl} {C}_{t r} ( {T}_{cl} - {T}_{a i r} ) ]$$

7. The system according to claim 6, wherein the control unit controls the power supplied to the heating member so that the sensor body is kept at a body temperature Tₛₑₙ which is substantially equal to the clothing surface temperature Tel, and PMV and PPD values are measured.

8. The system according to claim 4, the control unit being adapted to compare the total heating power supplied to the body for keeping the body at the predetermined temperature, with the heat flux measured by the reflective sensor and the heat flux measured by the absorptive sensor of each of the six or more sensor pairs.

9. The system according to claim 3, the control unit heating the body temperature Tₛₑₙ to the operative temperature Tₒₚₑᵣₐₜᵢᵥₑ thereby determining the operative temperature Tₒₚₑᵣₐₜᵢᵥₑ:
$${Φ}_{radiation} + {Φ}_{convection} = 0 at {T}_{sen} = {T}_{operative} :$$
with Φ_{radiation} and Φ_{convection} sums of radiative and convective heat fluxes to the sensor body weighted with the applicable area factors.

10. Method of determining a comfort value, the method comprising the steps of:
providing a heat flux sensor having a body with six or more sensor pairs, each sensor pair consisting of one absorptive sensor for measuring a combined radiative and convective heat flux and one reflective sensor for substantially measuring a convective heat flux, a heating member that is in heat conducing contact with the body, a temperature sensor thermally coupled with the body for measuring the body temperature Tₛₑₙ, and a humidity sensor connected to the control unit for determining a humidity pₐ of the ambient air, and an ambient temperature sensor connected to the control unit for measuring an ambient temperature Tₐᵢᵣ,
operating the heating member for heating of the body, to control the body temperature Tₛₑₙ to an estimated clothing surface temperature T_{cl} determining the temperature of the body, Tₛₑₙ,
determining for each of the six or more sensor pairs from the measurements of the reflective sensor a convective heat flux Φ_{convection},
determining for each of the six or more sensor pairs a radiative heat flux Φ_{radiation} by subtracting the measurements of the absorptive sensor and of the reflective sensor,
entering values of M metabolic rate, W work, I_{cl}, clothing insulation, f_{cl} clothing surface area factor and
determining the comfort value based on:
the measured humidity pₐ of the ambient air,
the measured air temperature, Tₐᵢᵣ,
the body temperature, Tₛₑₙ,
for each of the six or more sensor pairs the radiative heat flux Φ_{radiation}, and the convective heat flux Φ_{convection}.
using mathematical corrections such as projected area factors valid for a person at the position of the sensor.

11. The method according to claim 10, wherein the comfort value comprises a predicted mean vote PMV which is a function of parameters including a metabolic rate M, that can be expressed in W/m², the effective mechanical power W, that can be expressed in W/m², clothing insulation I_{cl}, that can be expressed in K·m²/W, the air temperature Tₐᵢᵣ, a mean radiant temperature T_{rad}, that can be expressed in C, the ambient air velocity vₐᵢᵣ, that can be expressed in m/s and the measured humidity pₐ that can be expressed by water vapor partial pressure in Pa, which parameters are related by:
$$P M V = [ 0.303 {e}^{( - 0.036 M )} + 0.028 ] \times [ [ ( M - W ) - 3.05 \times {10}^{- 3} [ 5733 - 6.99 ( M - W ) - {p}_{a} ] - 0.42 [ ( M - W ) - 58.15 ] - 1.7 \times {10}^{- 5} M ( 5867 - {p}_{a} ) - 0.0015 M ( 34 - {T}_{a i r} ) - 3.96 {10}^{- 8} {f}_{cl} ( {( {T}_{cl} + 273 )}^{4} - {( {T}_{r a d} + 273 )}^{4} ) - {f}_{cl} {C}_{t r} ( {T}_{cl} - {T}_{a i r} ) ] ,$$
Wherein values of M, W, I_{cl}, f_{cl} are entered by the user, and f_{c}l is a clothing surface area factor, and T_{cl} the clothing surface temperature
$${T}_{cl} = 3 5.7 - 0.028 ( M - W ) - {I}_{cl} [ 3.96 {10}^{- 8} {f}_{cl} ( {( {T}_{cl} + 273 )}^{4} - {( {T}_{r a d} + 273 )}^{4} ) + {f}_{cl} {C}_{t r} ( {T}_{cl} - {T}_{a i r} ) ]$$

12. Method of determining an operative temperature Tₒₚₑᵣₐₜᵢᵥₑ, the method comprising the steps of:
providing a heat flux sensor having a body with six or more sensor pairs, each sensor pair consisting of one absorptive sensor for measuring a combined radiative and convective heat flux and one reflective sensor for substantially measuring a convective heat flux, a heating member that is in heat conducing contact with the body, a temperature sensor thermally coupled with the body for measuring the body temperature Tₛₑₙ,
operating the heating member for heating of the body,
determining the temperature of the body, Tₛₑₙ,
determining for each of the six or more sensor pairs from the measurements of the reflective sensor a convective heat flux Φ_{convection},
determining for each of the six or more sensor pairs a radiative heat flux Φ_{radiation} by subtracting the measurements of the absorptive sensor and of the reflective sensor, controlling the sensor temperature Tₛₑₙ to a temperature Tₒₚₑᵣₐₜᵢᵥₑ for which
$${Φ}_{radiation} + {Φ}_{convection} = 0$$
with Φ_{radiation} and Φ_{convection} sums of radiative and convective heat fluxes to the sensor body weighted with the applicable area factors,
determining Tₒₚₑᵣₐₜᵢᵥₑ.

13. System comprising the heat flux sensor of claim 2, a control unit and an ambient temperature sensor connected to the control unit for measuring an ambient temperature Tₐᵢᵣ, the control unit being adapted for:
operating the heating member for heating of the body,
determining or controlling of the temperature of the body, Tₛₑₙ
determining from the measurements of the reflective sensor of each of the six or more sensor pairs a convective heat flux Φ_{convection},
determining a radiative heat flux Φ_{radiation} by subtracting the measurements of the absorptive sensor and of the reflective sensor of each of the six or more sensor pairs, determining a convective heat transfer coefficient Cₜᵣ based on Φ_{convection}, Tₐᵢᵣ and Tₛₑₙ or based on the power supplied to the heating member at several sensor body temperatures Tₛₑₙ,
determining an ambient air velocity vₐᵢᵣ, based on the heat transfer coefficient Cₜᵣ, and
determining the radiative temperature T_{rad} from Tₛₑₙ and Φ_{radiation}.

14. The system according to claim 4, further comprising a humidity sensor connected to the control unit for determining a humidity pₐ of the ambient air, the control unit being adapted for determining a comfort value for a person at the position of the sensor, based on:
the measured humidity pₐ of the ambient air,
the measured air temperature, Tₐᵢᵣ,
the body temperature, Tₛₑₙ,
for each of the six or more sensor pairs, the radiative heat flux Φ_{radiation}, and the convective heat flux Φ_{convection}.
and using mathematical corrections such as projected area factors valid for a person at the position of the sensor.

15. The system according to claim 14, wherein the comfort value comprises a predicted mean vote PMV which is a function of parameters including a metabolic rate M, that can be expressed in W/m², the effective mechanical power W, that can be expressed in W/m², clothing insulation I_{cl}, that can be expressed in K·m²/W, the air temperature Tₐᵢᵣ, a mean radiant temperature T_{rad}, that can be expressed in C, the ambient air velocity v^{air}, that can be expressed in m/s and the measured humidity pₐ that can be expressed by water vapor partial pressure in Pa, which parameters are related:
$$P M V = [ 0.303 {e}^{( - 0.036 M )} + 0.028 ] \times [ [ ( M - W ) - 3.05 \times {10}^{- 3} [ 5733 - 6.99 ( M - W ) - {p}_{a} ] - 0.42 [ ( M - W ) - 58.15 ] - 1.7 \times {10}^{- 5} M ( 5867 - {p}_{a} ) - 0.0015 M ( 34 - {T}_{a i r} ) - 3.96 {10}^{- 8} {f}_{cl} ( {( {T}_{cl} + 273 )}^{4} - {( {T}_{r a d} + 273 )}^{4} ) - {f}_{cl} {C}_{t r} ( {T}_{cl} - {T}_{a i r} ) ] ,$$
Wherein values of M, W, I_{cl}, f_{cl} are entered by the user, and f_{cl} is a clothing surface area factor, and T_{cl} the clothing surface temperature:
$${T}_{cl} = 3 5.7 - 0.028 ( M - W ) - {I}_{cl} [ 3.96 {10}^{- 8} {f}_{cl} ( {( {T}_{cl} + 273 )}^{4} - {( {T}_{r a d} + 273 )}^{4} ) + {f}_{cl} {C}_{t r} ( {T}_{cl} - {T}_{a i r} ) ] \qquad [ 5 ]$$

16. The system according to claim 15, wherein the control unit controls the power supplied to the heating member so that the sensor body is kept at a body temperature Tₛₑₙ which is substantially equal to the clothing surface temperature T_{cl}, and PMV and PPD values are measured.

17. The system according to claim 16, the control unit being adapted to compare the total heating power supplied to the body for keeping the body at the predetermined temperature, with the heat flux measured by the reflective sensor and the heat flux measured by the absorptive sensor of each of the six or more sensor pairs.

18. The system according to claim 5, the control unit being adapted to compare the total heating power supplied to the body for keeping the body at the predetermined temperature, with the heat flux measured by the reflective sensor and the heat flux measured by the absorptive sensor of each of the six or more sensor pairs.

19. The system according to claim 6, the control unit being adapted to compare the total heating power supplied to the body for keeping the body at the predetermined temperature, with the heat flux measured by the reflective sensor and the heat flux measured by the absorptive sensor of each of the six or more sensor pairs.

20. The system according to claim 7, the control unit being adapted to compare the total heating power supplied to the body for keeping the body at the predetermined temperature, with the heat flux measured by the reflective sensor and the heat flux measured by the absorptive sensor of each of the six or more sensor pairs.
